(19) United States
(12) Patent Application Publication
(10) Pub. No.: US 2024/0390134 A1
(43) Pub. Date: Nov. 28, 2024

(54) METHODS AND IMPLANTABLE PROSTHESIS FOR ANATOMICAL RECONSTRUCTION AND/OR AUGMENTATION

(71) Applicant: Davol Inc., Warwick, RI (US)

(72) Inventors: Ian K. Parker, Bristol, RI (US); Talia J. D'Ambruoso, Sunnyvale, CA (US); Peter Maughan Crapo, North Kingstown, RI (US); Cameron Michael Correia, Warwick, RI (US); Angel Pagan-Ortiz, Providence, RI (US); Jonathan Bruce Trexler, Rehoboth, MA (US); Evans Kipyego, Warwick, RI (US); Jeremy Griffin, North Attleboro, MA (US); Korel Cudmore, Acushnet, MA (US)

(73) Assignee: Davol Inc., Warwick, RI (US)

(21) Appl. No.: 18/693,371

(22) PCT Filed: Sep. 23, 2022
(86) PCT No.: PCT/US2022/044517

§ 371 Date: Mar. 19, 2024

Related U.S. Application Data
(60) Provisional application No. 63/247,860, filed on Sep. 24, 2021.

(51) Int. Cl. A61F 2/12

(52) U.S. Cl. CPC A61F 2/12; A61F 2250/0063

(57) ABSTRACT

An implantable prosthesis including a tissue infiltratable body of biocompatible material, the prosthesis having a body with a three-dimensional configuration to augment and/or reconstruct an anatomical shape of a human breast. The body includes a plurality of body segments stacked on top of one another about a longitudinal axis extending in a direction from a proximal end to a distal end of the prosthesis. The proximal end is configured to be positioned against facia in an anatomical space.

### FIELD

The present disclosure relates to an implantable prosthesis, and more particularly to a prosthesis for breast reconstruction and/or augmentation.

### BACKGROUND

Breast reconstruction is primarily performed following breast cancer diagnosis and surgical treatment. However, a growing number of patients are choosing breast reconstruction as a prophylactic option in response to genetic testing results which may indicate an individual being at high risk for breast cancer.

Breast reconstruction can be generally categorized as autologous and non-autologous. For autologous reconstruction, a patient's own tissue is harvested from another part of their body and then used to reconstruct the breast. For non-autologous reconstruction, an artificial implant, such as a saline, silicone or gel implant, is employed to reconstruct the breast mound.

Autologous reconstruction generally involves harvesting a tissue flap from the abdominal region of a patient. This procedure can maintain vascular supply to the patient's tissue, and generally provides an aesthetically pleasing outcome for the patient. However, such a procedure can be time consuming, with possible microsurgery to reconnect the vascular supply, and require a relatively longer recovery time. It can also create functional deficits and weakness in the area from which the tissue has been removed. This technique may not be available to some patients who lack belly volume or cannot afford a reduction of muscle mass.

Non-autologous reconstruction, which involves a wide majority of breast reconstructive procedures, may employ single stage or intermediate reconstruction procedures, or dual stage reconstruction procedures. A mastectomy and reconstruction of the breast can be performed at the same time (single stage) or staged over multiple procedures (dual stage). In each procedure, a breast implant is typically placed below the pectoral muscle, i.e., sub-pectoral, to mask the implant from being seen through the skin and cover the relatively stiff implant with muscle.

In a single stage procedure, breast tissue is completely dissected and removed after a small incision is made under the breast. The pectoral muscle is subsequently detached at its lower end and the sub-muscular plane is developed to create a sub-pectoral pocket with sufficient size to accommodate the implant. An acellular dermal matrix (ADM) is typically employed to reattach the muscle and add reinforcement under the implant.

Single stage procedures generally do not provide much control over the cosmetic outcome because it cannot be adjusted over time. These procedures could also potentially result in tissue necrosis should the implant be too large for the size of the sub-muscular pocket.

In a dual stage procedure, the initial surgical stage is similar to the single stage procedure. However, rather than placing an implant into the sub-muscular pocket, an ADM is initially placed in the pocket and followed by placement of a tissue expander. The ADM is manipulated as needed to accommodate the tissue expander and then fixated into place. Following the initial surgical stage, the tissue expander is filled over multiple post-surgical office visits to slowly expand the space below the pectoral muscle to create a pocket. Once a sufficiently sized pocket is formed, typically six months after the initial procedure, a second surgical procedure is performed to remove the expander and insert the breast implant in the sub-muscular pocket created by the expander.

A more recent trend in breast reconstruction involves pre-pectoral placement of an implant on top of the pectoral muscle to avoid creation of a sub-muscular pocket. During such procedures, the implant is typically wrapped completely with ADM rather than using the ADM as a sling which only partially covers the implant.

It has been reported that the breast is shaped by a three-dimensional, fibrofatty fascial system. Two layers of this system surround the corpus mammae and fuse together around and anchor it to the chest wall in a structure identified as the circum-mammary ligament (CML). The CML, which defines the perimeter of the breast, is a 3D, roughly circular structure composed of superficial fascia collagen fibers that encase a ring of fat and attach it to the deep fascia of the chest, as a circular zone of adherence.

It is an object of the disclosure to provide methods and a prosthesis for augmenting and/or reconstructing a breast.

### SUMMARY

The present disclosure relates to methods and an implantable prosthesis for augmenting and/or reconstructing a breast.

According to one embodiment, an implantable prosthesis includes a tissue infiltratable body having a plurality of voids configured to receive fat and/or tissue, the body including a proximal end and a distal end spaced from the proximal end. The body includes a plurality of three-dimensional (3D) body segments arranged in a stacked configuration along a longitudinal axis extending in a direction from the proximal end to the distal end of the body, wherein each of the 3D body segments include one or more outwardly facing voids.

According to another embodiment, an implantable prosthesis includes a tissue infiltratable body having a plurality of voids configured to receive fat and/or tissue, the body including a proximal end and a distal end spaced from the proximal end. The body includes a plurality of three-dimensional (3D) body segments stacked on top of one another along a longitudinal axis extending in a direction from the proximal end to the distal end of the body, wherein each of the plurality of body segments is a toroidal shape. Each of the plurality of body segments is in direct contact with an adjacent body segment.

According to another embodiment, a method of fabricating an implantable prosthesis having a plurality of voids configured to receive fat and/or tissue is disclosed. The method includes the acts of (a) stacking a first three-dimensional (3D) body segment along a longitudinal axis of a body extending in a direction from a proximal end to a distal end of the body, (b) stacking a first two-dimensional (2D) body segment along the longitudinal axis of the body, and (c) stacking a second 3D body segment along the longitudinal axis of the body, and (d) attaching the first 3D body segment. The first 2D body segment, and the second 3D body segment together, wherein the first 2D body segment is positioned in between the first 3D body segment and the second 3D body segment.

It should be appreciated that the foregoing concepts, and additional concepts discussed below, may be arranged in any suitable combination, as the present disclosure is not limited in this respect.

The foregoing and other aspects, embodiments, and features of the present teachings can be more fully understood from the following description in conjunction with the accompanying drawings.

### BRIEF DESCRIPTION OF DRAWINGS

Aspects of the disclosure are described below, by way of example, with reference to the accompanying drawings, and wherein:

FIGS. 1A-1F are schematic illustrations of a breast reconstruction procedure;

FIG. 2 is a perspective view of an implantable prosthesis for breast reconstruction and/or augmentation according to an embodiment of the present disclosure;

FIG. 3 is an exploded view of body segments of the prosthesis of FIG. 2;

FIG. 4 is a side view of the prosthesis of FIG. 2;

FIG. 5 is a cross-sectional side view of a prosthesis according to another embodiment;

FIG. 6 shows body segments of an implantable prosthesis according to another embodiment;

FIG. 7 is an exploded view of body segments of an implantable prosthesis according to another embodiment;

FIG. 8 is a side view of an implantable prosthesis according to another embodiment;

FIG. 9 is a side view of an implantable prosthesis according to still another embodiment;

FIG. 10 is a side view of an implantable prosthesis according to another embodiment;

FIGS. 11A-11D illustrate formation of a body segment with a fanned arrangement, with FIG. 11A being a top view of an unfolded sheet, FIG. 11B being a side view of an accordion folded sheet, FIG. 11C being a top view of a folded sheet in collapsed arrangement, and FIG. 11D being a top view of a fanned arrangement of the body segment;

FIG. 12 is a top view of a prosthesis having body segments with fanned arrangements according to one embodiment;

FIG. 13 is a perspective view of a honeycomb structure of a body segment according to one embodiment;

FIG. 14A is a top view of a stack of sheets used to form the honeycomb structure shown in FIG. 13;

FIG. 14B is a side view of the stack of sheets of FIG. 14B;

FIG. 15 is a perspective view of an implantable prosthesis according to another embodiment;

FIG. 16 is a perspective view of an implantable prosthesis according to another embodiment, with a base shown removed from a body of the prosthesis;

FIG. 17 is an exploded side view of body segments of the implantable prosthesis of FIG. 12;

FIG. 18 is a cross-section view of the implantable prosthesis of FIG. 17;

FIG. 19 is a bottom perspective view of a body segment of the implantable prosthesis of FIG. 17; and

FIG. 20 is a perspective view of a portion of an implantable prosthesis according to one embodiment.

### DESCRIPTION

The present disclosure is directed to an implantable prosthesis particularly suited for augmenting and/or reconstructing a breast. However, the prosthesis may be suitable for mending anatomical defects in, and weaknesses of, soft tissue and muscle walls or other anatomical regions. For purposes herein, the phrase “mending a defect” includes acts of repairing, augmenting, and/or reconstructing a defect and/or a potential defect.

For case of understanding, and without limiting the scope of the disclosure, the prosthesis is described below particularly in connection with breast reconstruction. It should be understood, however, that the prosthesis is not so limited and may be employed in other anatomical procedures, as should be apparent to one of skill in the art. For example, and without limitation, the prosthesis, or aspects of the prosthesis, may be employed for hernias, chest or abdominal wall reconstruction, or large defects, such as those that may occur in obese patients. The prosthesis may include one or more features, each independently or in combination, contributing to such attributes.

As will be described further below, the prosthesis may have particular application with pre-pectoral breast reconstruction procedures.

### Pre-pectoral Breast Reconstruction

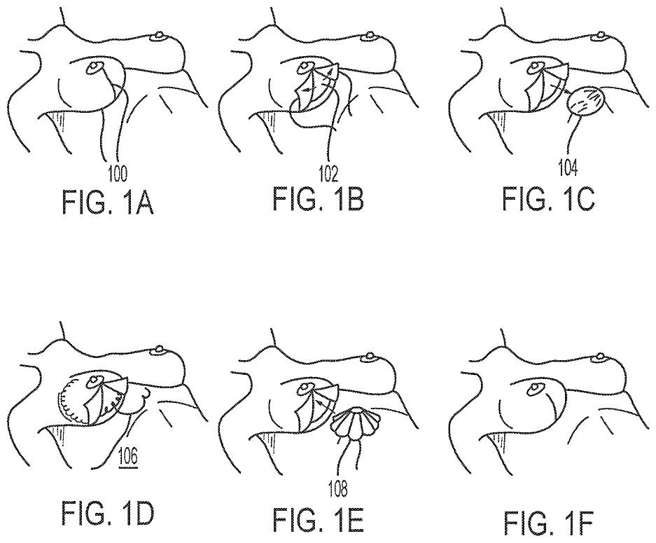

One embodiment of a pre-pectoral breast reconstruction procedure is illustrated in FIGS. 1A-1F.

As illustrated in FIG. 1A, an initial incision 100 may be made to form tissue flaps 102 on a lower portion of the breast. The flaps 102 may be spread apart to provide access to the breast tissue and permit removal of a tumor or other growth, as illustrated in FIG. 1B. It is desirable to preserve skin and the nipple arcola complex, when possible.

As illustrated in FIG. 1C, the corpus mammae 104 may be removed, along with the bases of the Cooper's ligaments. Thereafter, as illustrated in FIG. 1D, the circum-mammary ligament (CML) 106 may be tightened to reestablish a desirable diameter base for the breast. For example, the CML may be tightened to correspond with the breast's pre-op diameter base, or tightened even more should the CML have been stretched and loosened over time due to aging and/or other factors.

As illustrated, the CML may be tightened using a purse string suturing technique in which a running suture is placed around and/or through the CML and then pulled upwardly to cinch-up the tissue and tighten the base. However, other procedures are contemplated for reestablishing the base of the breast.

Once the CML has been tightened, a prosthetic implant 108 may be inserted, as illustrated in FIG. 1E, into the anatomical cavity created by the removal of the corpus mammae. Prior to insertion, the implant may be coated with a fat graft which has been lipo-aspirated from the patient. In some embodiments, the fat graft may help soften the prosthesis and/or provide seeding for new fat and/or tissue being formed in and about the prosthesis. The fat graft may also reduce the potential for fluid to fill the space created by the removal of tissue during the procedure. However, it is to be appreciated that the implant does not need to be coated with a fat graft prior to insertion.

If desired, the fat graft may be harvested from the patient using standard lipo-aspiration techniques. The aspirated fat may be processed on site to remove oils and provide a more purified fat for the procedure. The processed fat may be applied to various surfaces and within various voids of the prosthesis using a syringe or similar device, although other techniques are contemplated for coating the prosthesis.

Once inserted, the implant may be secured to the CML using sutures or other fasteners placed along the base of the device. Following implantation of the prosthesis, the incision may be closed without tension, as illustrated in FIG. 1F.

Following the initial reconstruction procedure, fat grafting into the breast may be performed with one or more procedures over time to achieve a desired shape and/or feel for the breast and/or symmetry between both breasts. For example, in some embodiments, process fat may be injected into the breast and into the various voids of the prosthesis.

### Prosthesis and Fabrication Concepts

The disclosure is more particularly directed to an implantable prosthesis, such as for breast reconstruction and/or augmentation. According to one aspect, the implantable prosthesis may include a tissue infiltratable body of a biocompatible material that is placed within an anatomical space.

In some embodiments, the body may have a three-dimensional (3D) configuration, such as to reconstruct and/or augment the anatomical shape of a breast. The prosthesis may be configured to encourage the ingrowth of fat and/or tissue to fill open spaces within and about the prosthesis. The prosthesis may employ a structure having a plurality of voids including, but not limited to, chambers, cavities, channels, openings, pockets, and/or pores, to allow fat and/or tissue to fill and pass through the structure and fill-out the reconstructed or augmented breast. The overall desired structure of the prosthesis may employ various constructs for its fabrication in an efficient manner.

In some embodiments, the prosthesis may include a body fabricated using a plurality of body segments which can be assembled to create the desired configuration of the of the body (e.g., desired shape, number of voids, and/or compressibility of the body). In some embodiments, each body segment may include a two-dimensional (2D) configuration. In such embodiments, the 2D body segments may be attached to one another and/or to another suitable portion of the prosthesis to form the 3D body having one or more cavities for receiving fat and/or tissue. The 2D body segments also may be transformed into a 3D configuration before attaching the body segments to one another and/or to another suitable portion of the body. For example, in some embodiments, a planar body segment may be folded, bent, or otherwise shaped prior to attachment to another body segment and/or another portion of the prosthesis. In this manner, the individual segments may be fabricated in a relatively less complex manner and then assembled to create the desired arrangement (e.g., shape and/or number of voids) of the prosthesis. In some embodiments, the prosthesis may include a combination of 2D and 3D body segments.

In still other embodiments, the body segments may be formed (e.g., 3D printed or molded) to have a 3D arrangement, with the 3D body segments being attached to one another. In some embodiment, such 3D body segments also may be attached to one or more 2D body segments. In some embodiments, the body may be configured to provide the prosthesis with a desired amount of resilience and support.

In some embodiments, the body segments may be stacked on top of one another along a longitudinal axis of the body. For example, in some embodiments, each body segment may form a tier of the body. In some embodiments, multiple 3D body segments may be stacked on top of one another to form the 3D body. In other embodiments, 2D and 3D body segments may be stacked on top of one another to form the 3D body. For example, 3D and 2D body segments may be alternated along the longitudinal axis to form the 3D body. In still other embodiments, body segments may be stacked on top of one another in a nested configuration to form the 3D body.

In some embodiments, the body may include a proximal end, such as a base or bottom surface for placement against fascia, such as the pectoral muscle. In some embodiments, the base may be planar. In some embodiments, the base may have a generally rounded shape including, but not limited to, a convex shape. Such a configuration may facilitate positioning and placement of the prosthesis against the pectoral muscle and within the circum-mammary ligament, which may establish the position of the prosthesis on the chest wall. In this manner, the interaction between the implant and the anatomy may create a ball and socket like arrangement.

The prosthesis also may include a distal end that is spaced from the proximal end. For example, in some embodiments, the distal end may include the top of the prosthesis. In some embodiment, the longitudinal axis may extend from the proximal end to the distal end of the prosthesis. In some embodiments, the plurality of body segments may extend from the proximal end (e.g., bottom surface or base) of the prosthesis to the distal end of the prosthesis. In such embodiments, the body segments may be arranged to form voids for receiving the fat and/or tissue.

In some embodiments, the voids may be defined by a space in between adjacent body segments. In some embodiments the voids may extend in a direction away from the longitudinal axis and away from the outer periphery. In other embodiments, the voids may extend in a direction away from the longitudinal axis and away from a proximal end of the prosthesis. For example, in some embodiments, the proximal end may be placed against facia, with the voids extending in a direction towards the facia. In other embodiments, the body segments may be formed of a porous material, such as a mesh, with the voids being defined by the pores of the material. In such embodiments, fat and/or tissue may travel through and/or into the pores.

In some embodiments, the body segments may be attached to one another via stitching (e.g., sutures), ultrasonic welding, or another suitable attachment method. In other embodiments, the prosthesis may include a hub to which one or more body segments are attached. For example, the prosthesis may include a hollow core structure. In some embodiments, stacking of the body segments themselves may defined the hollow central core. In some embodiments, fat and/or tissue may travel in and around the hollow cores. In some embodiments, the prosthesis may include an exterior skeleton to support the prosthesis. For example, in some embodiments, the prosthesis my include one or more exterior ribs to provide support (e.g., stability) to the prosthesis.

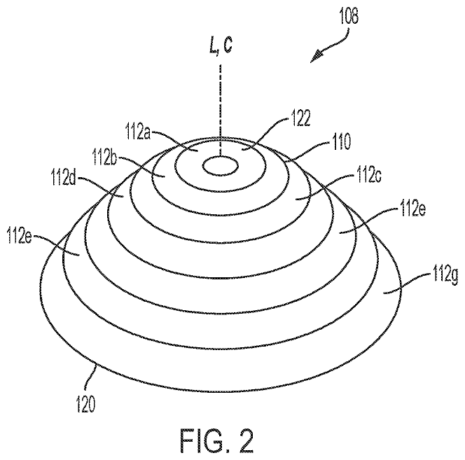

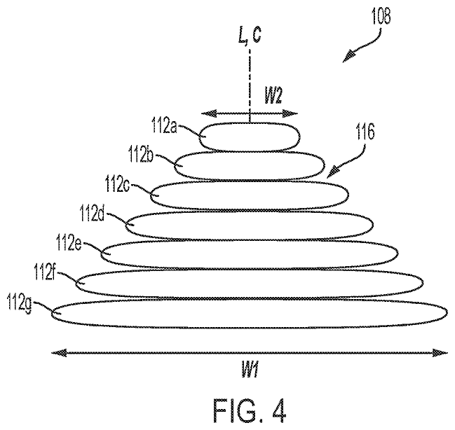

Turning now to the figures, FIGS. 2-4 illustrate an implantable prosthesis 108 according to one embodiment of the present disclosure. As shown in these figures, the prosthesis may include a body with a plurality of 3D body segments 112a-112g coupled to one another to form the prosthesis body. In some embodiments, the body segments may form one or more voids 116 for receiving fat and/or tissue, for example, to fill-out a reconstructed or augmented breast. For example, as shown in FIG. 4, voids may be defined by the space between adjacent body segments, such as near the outer periphery.

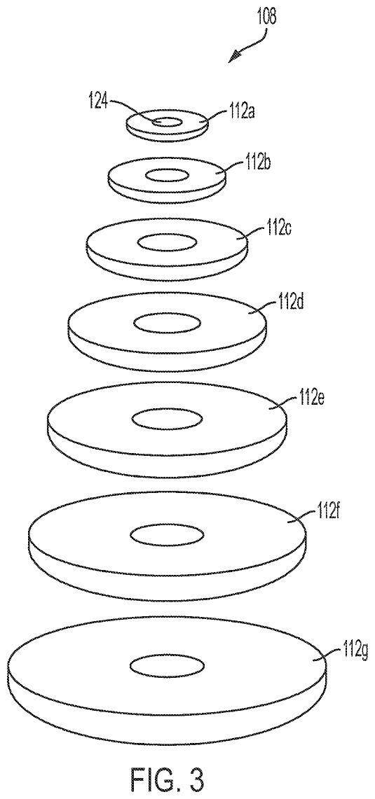

The prosthesis may include any suitable number of body segments, such as to vary the size and arrangement of the prosthesis. For example, in some embodiments, the prosthesis may include seven body segments, as shown in FIGS. 2 and 3. The prosthesis also may include, two, three, four, five, six, or more than seven body segments in other embodiments.

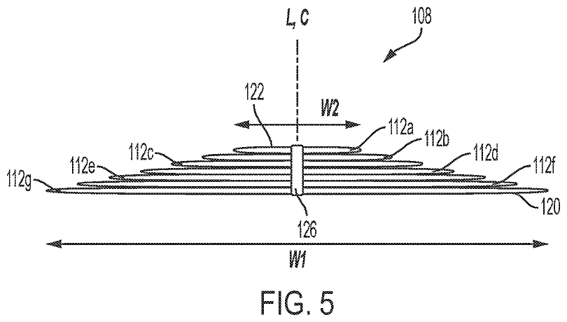

As will be appreciated, the shape and/or size of each body segment may be the same or may vary depending upon the desired configuration (e.g., 3D structure) of the prosthesis. In some embodiments, for example, the thickness or height of each layer may vary depending upon the desired size of the prosthesis (see, e.g., FIGS. 4 and 5 which show prostheses with body segments having different thicknesses). In other embodiments, the thicknesses of each body segment may be the same in the prosthesis, although the thickness of the body segments may vary in the prosthesis.

As shown in FIGS. 2 and 3, the body segments may be arranged in a stacked configuration. For example, in some embodiments, the body segments may be stacked on top of one another along the longitudinal axis L of the prosthesis. In some embodiments, as shown in these views, adjacent body segments may be placed in direct contact with one another. As will be appreciated, if desired, one or more layers also may be spaced apart from each other axially along the longitudinal axis. In some embodiments, the longitudinal axis may extend between a proximal end 120 and a distal end 122 of the prosthesis. In some embodiments, as shown in FIG. 3, the longitudinal axis may define the central axis C of the prosthesis.

In some embodiments, as shown in FIGS. 2-5, the body segment may be toroidal in shape and have an inner opening 124. In some embodiments, each body segment may include an opening in a central portion of the body segment. In some embodiments, the openings of the body segments may be aligned with one another. In some embodiments, the aligned openings may form a hollow core along the longitudinal axis of the prosthesis. In such embodiments, the body segments may be attached to one another (e.g., via sutures or ultrasonic welding). In other embodiments a central hub 126 (see, e.g., FIG. 5) may extend through each of the aligned openings along the longitudinal axis (see FIG. 3). In such embodiments, each body segment may be attached to the central hub.

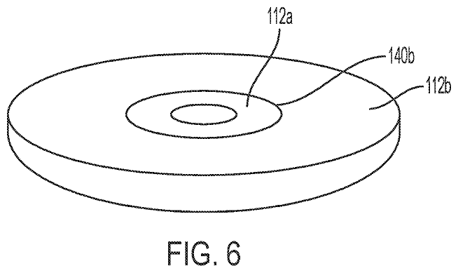

In some embodiments, as illustrated in FIG. 6, multiple body segments, such as multiple annular or toroidal segments, may be used to form a single tier of the body. For example, and without limitation, a first body segment may be positioned within an opening of an outer annular or toroidal structure. As will be appreciated, the shape of the first and second body segments may be the same (e.g., annular or toroidal), with the first body segment being smaller than the second body segment. The shape of the first and second body segments also may differ in some embodiments. In some embodiments, the thickness of the first and second body segments may be the same, although the thicknesses may differ in other embodiments. The first and second body segments may be joined together via any suitable manner (e.g., sutures and/or ultrasonic welding).

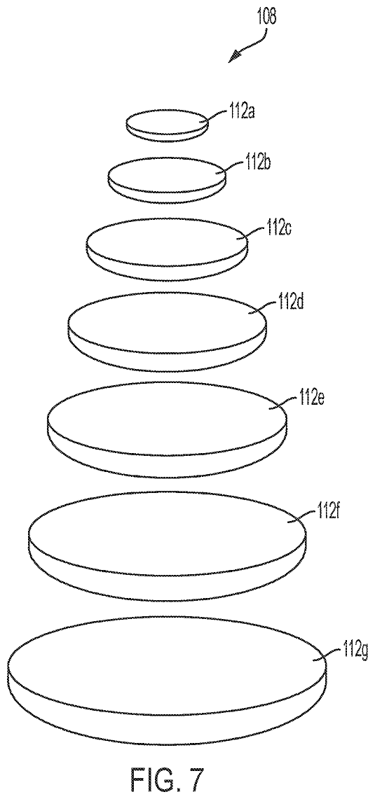

The body segments also may have other suitable shapes in other embodiments. For example, in some embodiments, one or more body segments may be square, triangular, rectangular, other polygonal, or other suitable shape. In some embodiments, the body segments may be formed without an opening in a central portion of the body segment (see, for example, the body segments in FIG. 7). As will be appreciated, the body segments may be formed in any suitable manner. For example, in some embodiments, the body segments may be formed (e.g., molded or 3D printed) to have a 3d configuration, such as the 3D toroidal shape.

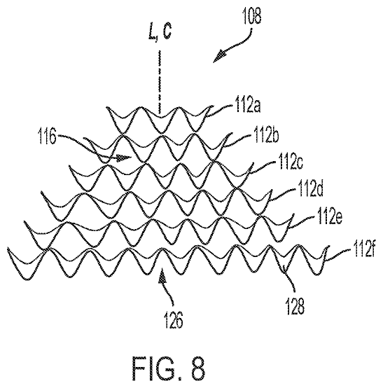

As shown in FIG. 8, in some embodiments, the 3D body segments may be configured with a plurality of pleats extending about and in an outward radial direction from a longitudinal axis of the prosthesis. In one embodiment, each body segment may be formed by a folded layer of material which is expanded in a fan-like manner about an axis. As shown in FIG. 8, when the body segments are assembled in the stacked configuration, the prosthesis includes a plurality of voids 116 for receiving fat and/or tissue. In such embodiments, the voids may be located between the pleats of the adjacent body segments.

As will be appreciated, the size and/or shape of the pleats may be varied to provide the implant with a desirable amount of stiffness and/or resilience. In some embodiments, the shape and/or size of the pleats may vary from body segment to body segment. The shape and/or size also may vary within the same body segment. For example, in some embodiments, a size of the pleats may increase in an outward radial direction from the longitudinal axis of the prosthesis. In some embodiments, as shown in FIG. 8, the pleats may be substantially U-Shaped. The pleats may have other suitable shapes (e.g., V-shaped) in other embodiments.

As shown in FIG. 8, in some embodiments, the prosthesis may include body segments with the same configuration directly coupled to one another. For example, as shown in this view, pleated body segments 112a-112f may be directly coupled to one another in a stacked configuration in some embodiments. As shown in FIGS. 3 and 4, a prosthesis also may have all body segments that are toroidal or annular in shape.

The body segments also may have different configurations relative to one another. For example, in some embodiments, the 3D arrangement of the body segments may be varied along the longitudinal axis. In some embodiments, a pleated body segment may be stacked on top of a 3D toroidal or annular body segment, which is, in turn, stacked on top of another pleated body segment. The body segments also may include different shapes, different sizes, different thicknesses, and/or any desired combinations thereof.

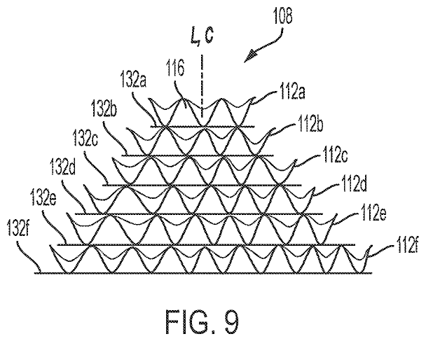

In other embodiments, the 3D body segments may be separated from one another by a planar body segment. For example, as shown in FIG. 9, a planar body segment 132a-132f may be positioned in between each pair of adjacent pleated body segments 12a-112f. In such embodiments, voids 116 may be formed between the planar body segment and the adjacent pleated body segment. In some embodiments, a planar body segment (e.g., 132f) may form a proximal or bottom surface to be positioned against fascia. In some embodiments, as shown in these views, a height of the 3D body segment may be greater than a height of the planar 2D body segment.

As will be appreciated, a planar body segment need not be positioned between each adjacent pair of body segments. For example, in some embodiments, planar sheets may be positioned between only a subset of the adjacent pairs of body segments. In some embodiments, the size (e.g., diameter) and shape of a planar body segment may be the same as that of an adjacent pleated body segment. In other embodiments, the size of the planar body segment may differ from that of an adjacent pleated segment.

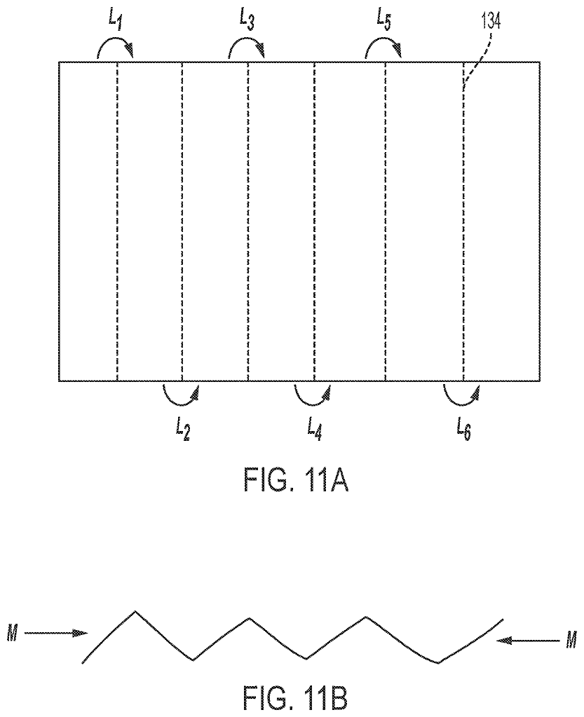

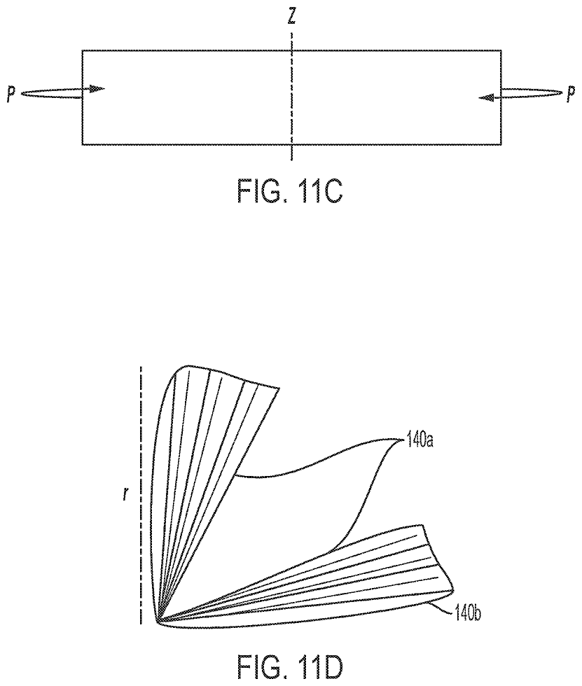

In still another embodiment, as shown in FIG. 12, each body segment may be formed from multiple sheets that have each been folded into a fan shape (see FIG. 11D) and thereafter attached to one another. FIG. 11A shows a sheet of material used to form the body segment. As shown in this view, the sheet may include multiple fold lines 134 along which the sheet may be folded (see arrows L₁-L₆) in an alternating manner to form pleats. For example, an accordion folding method may be used to form a sheet with parallel pleats. As shown in FIG. 11B, the pleats may be substantially V-shaped in some embodiments, with voids being formed between the pleats.

In some embodiments, the pleated sheet may be collapsed or folded together (see arrows M in FIG. 11B). In such embodiments, the pleated panels may be stacked on top of one another. As shown in FIG. 11C, first and second ends of the stacked and folded sheet may be moved towards each other (see arrows P), with the stacked and folded sheet being folded about axis Z to form the fan arrangement shown in 11D. As will be appreciated, in this fan arrangement, a first side of the folded sheet may form the inner potion 140a of the fan, while the second, opposite side of the folded sheet may form the outer portion 140b of the fan. In some embodiments, the inner portions of the sheet may be attached together, such as via spot welding. In some embodiments, the outer portions 140b of adjacent sheets may be attached together, such as via spot welding, to form the body segment (see FIG. 12).

As will be appreciated, the body segments of the present may be formed with the same diameter or with different diameters. Accordingly, sheets may be used to form fan arrangements having different radii r (see FIG. 11D), such as 2.5 cm, 3.75 cm, 5 cm, and 7.5 cm. In such embodiments, the body segment may be formed with a diameter of 5 cm, 7.5 cm, 10 cm, or 15 cm. As will be appreciated, multiple body segments may be stacked on top of one another to form the 3D prosthesis body. in other embodiments, the body segments may be separated via a planar body segment.

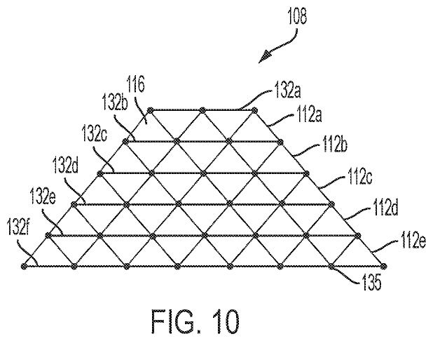

Turning back to FIG. 10, in some embodiments, each of the 3D body segments 112a-112e may be stacked on top of one another and separated via planar body segments 132a-132f. In such embodiments, the body segments may be attached to one another via ultrasonic spot welds 135. As will be appreciated, in other embodiments, the body segments may be directly coupled to one another, such as via spot welding or another suitable method.

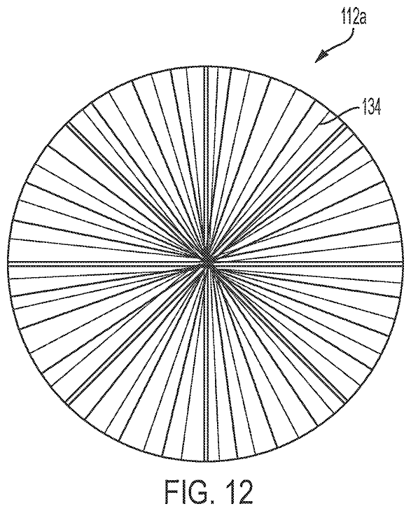

According to another embodiment, the body segments may include honeycomb structures (scc, e.g., FIG. 13). In such embodiments, a plurality of honeycomb structures may be arranged in a stacked configuration, similar to those described above. In some embodiments, the honeycomb-like structures may directly contact one another, although the honeycomb-like structures also may be separated with planar body segments. In one embodiment, a plurality of honeycomb structures may be arranged in a circumferential configuration about the central axis of the implant. Although described as having multiple stacked honeycomb structures, it will be appreciated that the body may include a single honeycomb structure.

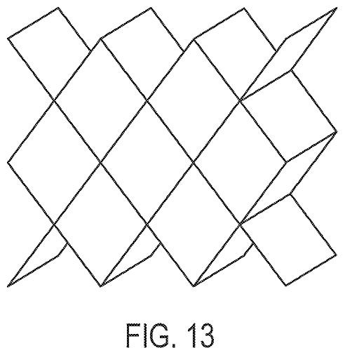

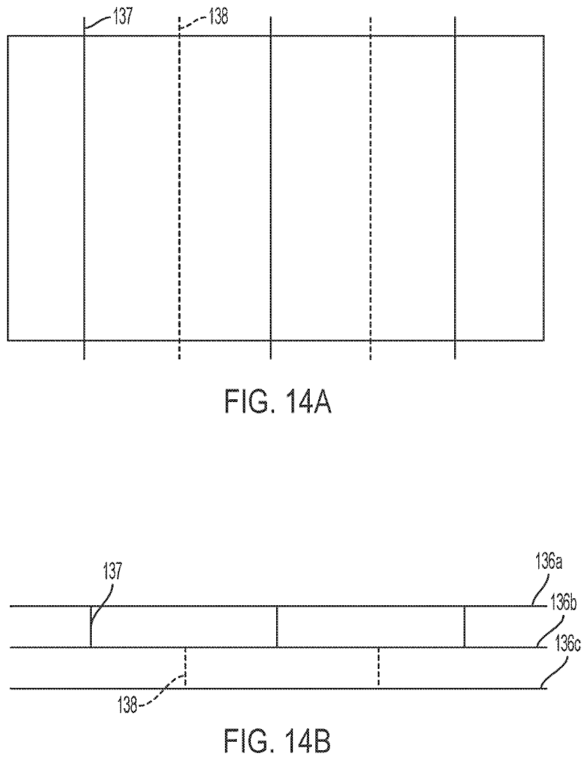

In some embodiments, the honeycomb structure may be formed by a plurality of layers of material that are stacked and joined together (see FIG. 13). FIG. 14A is a top view of a stack of planar sheets attached to each other to form the honeycomb structure. As illustrated in FIGS. 14A and 14B, the sheets may be joined using an alternating attachment scheme. For example, as illustrated by the solid lines labeled 137, a first sheet 136a may be joined to a second 136b, adjacent sheet via a first attachment pattern. As illustrated by the dashed lines labeled 138, the second and third sheets 136b, 136c may be joined together via a second attachment pattern. As shown in FIG. 14A, the first and second attachment patterns 137, 138 may alternate along a longitudinal axis of the stack of sheets.

In some embodiment, the first attachment pattern 137 may include multiple attachments (e.g., spot welds) spaced apart from each other along the width of the stacked sheets (e.g., along the solid lines labeled 137). The second attachment pattern 138 also may similarly include multiple attachments space apart from each other along the width of the stacked sheets (e.g., along the dashed lines labeled 138). In some embodiments, the attachments of the first pattern may be offset from the attachments of the second pattern. In some embodiments, pulling the sheets apart from one another (e.g., pulling the top sheet away from the bottom sheet in the stack) after creating the attachments may form the 3D honeycomb structure of FIG. 13, with voids being formed between adjacent sheets.

As will be appreciated, the layers may be joined to each other using any suitable attachment technique. For example, and without limitation, the layers may be joined together using stitches and/or ultrasonic welds. The density and/or size of the honeycomb voids may be varied by the number and/or spacing of the attachment locations between the layers. For example, the closer the attachment locations are placed together, the smaller and denser the voids, and the further apart the attachment locations are spaced, the larger and less dense the voids. If desired, a honeycomb structure may include regions having voids with different sizes and/or densities by varying the patterns of the attachment locations between the various layers of material.

In some embodiments, as shown in FIGS. 4 and 5, a width of the body segments may decrease in a direction from the proximal end of the prosthesis (see, e.g., width W1 of body segment 112g) to the distal end 120 of the prosthesis body (see, e.g., width W2 of body segment 112a). For purposes herein, a width of the body segment may include a distance from a first lateral side of the body segment to a second, opposite lateral side of the body segment. As will be appreciated, in embodiments in which the body segment is circular in cross-sectional shape, the width of the body segment may correspond to a diameter of the body segment.

In some embodiments, the width of the body segments may decrease in a tapered manner from the proximal end of the prosthesis body to the distal end of the prosthesis body, such as that shown in FIGS. and 5. The width of the body segments also may decrease in other suitable manners in other embodiments. In some embodiments, all of the body components may have the same width.

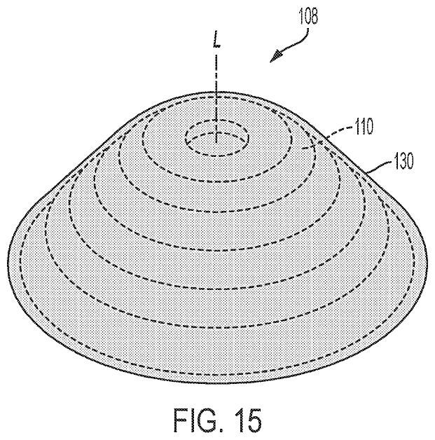

In some embodiments, as shown in FIG. 15, the prosthesis may include an outer layer 130 that covers the body. For example, in some applications, it may be desirable to provide a body with a generally smooth outer surface. In one embodiment, a sheet or relatively smooth material, such as a non-woven or other suitable material, may be attached about at least a portion of, if not the entire, outer peripheral surface of the body structures including, but not limited to, any of the structural arrangements descried above. In one embodiment, a layer of PHASIX material, available from Davol, Inc., may be used to cover the body structure. Other materials suitable for a cover structure may include, but are not limited to, collagen and ULTRAFOAM.

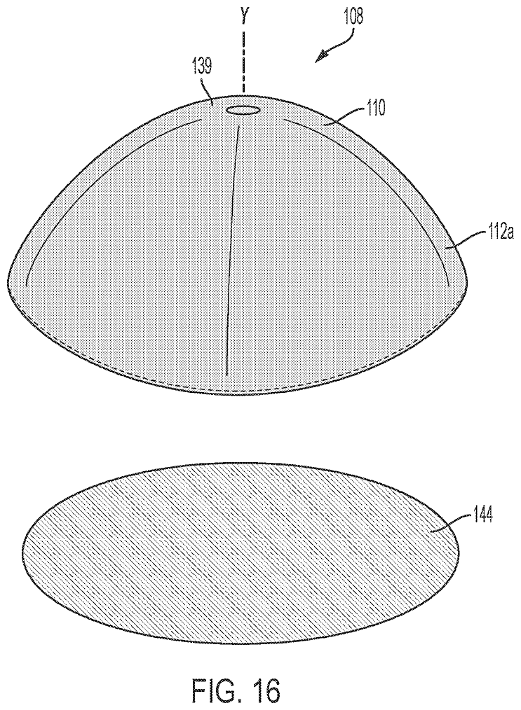

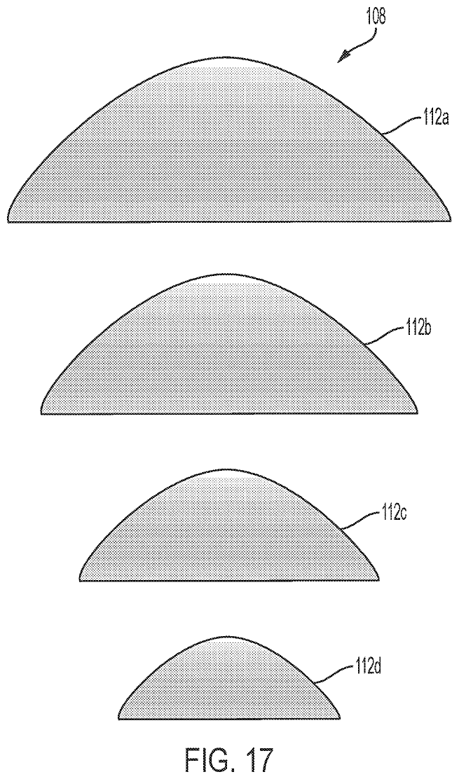

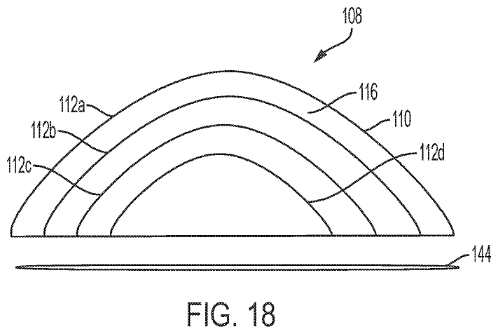

FIG. 16-19 show an implantable prosthesis according to another embodiment of the present disclosure. As shown in these figures, the prosthesis may include a body with a plurality of body segments 112a-112d (see FIGS. 17 and 18) stacked on top of one another in a nested configuration. For example, as shown in FIG. 14, a first body segment 112d may be nested within a second body segment 112c, which is nested within a third body segment 112b, which is nested within a fourth body segment 112a. As will be appreciated, the number of body segments can be increased and/or decreased to vary the size of the voids 116 formed between adjacent body segments and/or to vary the size of the prosthesis. As shown in FIG. 13, the size of the body segments may decrease in a direction from the distal end of the prosthesis towards the proximal end of the prosthesis.

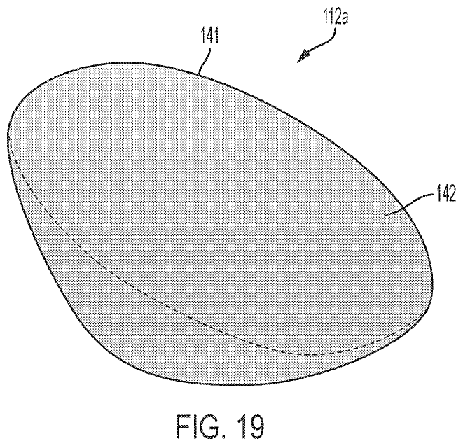

As shown in FIGS. 17 and 19, each of the body segments may have a 3D configuration. As shown in these figures, each body segments may include a first, closed end 139 and a second, open end 141 opposite the first end. In some embodiments, the second end is arranged to be positioned at or near the proximal end of the prosthesis. In some embodiments, the body segments may each define a cavity 142 for receiving a different body segment, fat and/or tissue. In some embodiments, the body segments may be substantially semi-hemispherical shaped, as shown in these views. The body segments also may have other suitable shapes (e.g., conical, frusto-conical, teardrop or pear shaped). As shown in FIG. 13, the body segments may all have the same shape in some embodiments. In other embodiments, the body segments may have different shapes.

In some embodiments, each body segment may be symmetric about a longitudinal axis Y of the body segment. In some embodiments, the longitudinal axis of the body segment may define the central axis of the body. In other embodiments, one or more body segments may be asymmetrical about the longitudinal axis. In such embodiments, one or more body segments may have the cross-sectional shape of a pear or half of a tear drop. As will be appreciated, the body segments may have other suitable shapes (e.g., conical, frusto-conical) in other embodiments.

As shown in FIGS. 12 and 14, in some embodiments, the prosthesis may include a base 144 attached to the prosthesis body for attaching the prosthesis to the body. As described herein, the base may form the proximal end of the prosthesis for placement against fascia. In some embodiments, the base may facilitate attachment of the prosthesis to the fascia. In some embodiments, the shape and size of the base may correspond to the shape of the proximal end of the prosthesis body. The shape and/or size of the base also may differ from that of the proximal end of the prosthesis body. For example, in some embodiments, the base may have a circular shape but may be larger than a circular shaped proximal end of the prosthesis body.

As will be appreciated, the base may have any suitable shape. For example, the base may have a substantially circular shape in some embodiments. The base also may have an oval, square, rectangular, triangular, other polygonal, or other suitable shape.

In some embodiments, the base may be attached to the body via sutures (e.g., stitching), ultrasonic welding, or any other suitable fastening technique as should be apparent to one of skill in the art. For example, in some embodiments, the base may be attached to the body via hook and loop fasteners. The base also may be attached to the body via other suitable portions of the prosthesis. For example, the base may be attached to a hub of the prosthesis, with each body segments also being attached to the hub.

In some embodiments, the base may be planar, as shown in FIG. 18. The base also may have other suitable configurations. For example, the bottom surface may have a generally rounded shape including, but not limited to, a convex shape. In some embodiments, such a curved shape may facilitate placement of the prosthesis against the fascia.

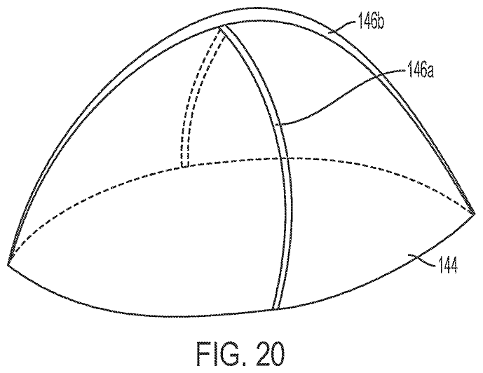

In some embodiments, the prosthesis may include an external skeleton structure to provide additional support to the prosthesis. In some embodiments, as shown in FIG. 20, the prosthesis may include first and second support ribs 146a, 146b which may extend over the prosthesis body. In some embodiments, each of the first and second ribs may be attached to the base 144 of the prosthesis. In some embodiments, each of the first and second ribs may extend from a first side (e.g., a first lateral side) of the proximal end of the prosthesis, over the distal end of the prosthesis, and to the second side (e.g., the second lateral side) of the prosthesis body. In some embodiments, each rib may be curved and shaped, for example, as an arch. The ribs may have other suitable shapes in other embodiments. In some embodiments, the ribs may cross over one another, such as at the distal end of the prosthesis. Although two support ribs are shown in FIG. 20, it will be appreciated that the prosthesis may have more or less ribs depending upon the desired arrangement of the prosthesis.

As will be appreciated in view of the present disclosure, the size of the prosthesis may be defined by the size and/or number of body segments. However, the desired size of a prosthesis may differ from patient to patient. Accordingly, for some applications, it may be desirable to adjust the size and/or bulk of the prosthesis by removing and/or adding material. For example, and without limitation, the size and/or bulk of the prosthesis may be reduced by removing one or more body segments (or portions thereof) from the prosthesis (or portions thereof). As will be appreciated, in some embodiments, bulk also may be added to the prosthesis by adding one or more body segments.

In some applications, the prosthesis may be pre-formed, with the body segments being assembled prior to a reconstruction procedure. For example, prior to reconstruction, the body segments may be attached to each other or to another suitable portion of the prosthesis, as described herein. In some embodiments, the pre-formed prosthesis may be trimmed to create a desired shape for implantation. For example, and without limitation, one or more body segments may be trimmed to create a customized implant shape. In such an example, the body segments may each be trimmed the same amount or may be trimmed different amounts depending upon the desired size of the prosthesis.

In other embodiments, additional body components may be added to the prosthesis to create the customized prosthesis shape. In such embodiments, the same body segments may be added to the prosthesis body, although different body segment may be added to the prosthesis body. In some embodiments, one or more body segments may be trimmed before attachment to the prosthesis body.

In some embodiments, the prosthesis may not be pre-formed but may instead be assembled in conjunction with a reconstruction procedure (e.g., prior to or during a procedure) to create the customized implant shape. In some embodiments, the prosthesis may include a modular design. For example, in some embodiments, the prosthesis may include a kit of modular components. In such embodiments, the prosthesis may include a kit of modular components. For example, the kit may include one or more body segments, a base and/or a hub. In such an example, a surgeon may select and attach one or more body segments together and/or to other suitable components to achieve the customized implant shape. In such an example, each body segment may have the same configuration or may different configurations. As with the above, the surgeon also may trim one or more of the body segments during assembly.

In some embodiments, the modular kit may include one or more pre-formed prosthesis bodies having different profiles and one or more bases. For example, the modular kit may include a low-profile body, a medium profile body, and a high-profile body. In some embodiments, the body segment may be selected by the surgeon based on the size of the cavity into which the prosthesis is being inserted. For example, a high-profile body may be chosen for a larger breast being reconstructed and/or augmented. The surgeon also may select a corresponding base and attach the base to the prosthesis body. In some embodiments, the base may be attached to the body via hook and loop fasteners, although other suitable attachment mechanisms may be used.

In some embodiments, the body segments in may be pre-formed before attachment to one another and/or to another suitable portion of the prosthesis. For example, the body may include a 2D configuration that is attached to the prosthesis. The body segment also may include a 2D configuration that is transformed into a 3D configuration before attaching to another body component, a base, or another suitable portion of the prosthesis body. In some embodiments, the 2D configuration may include a sheet which is corrugated, pleated, folded, or otherwise formed into the 3D configuration. The 3D configuration also may be formed (e.g., molded) via other suitable methods.

Various structural arrangements and/or manufacturing techniques may be employed to fabricate a relatively complex implant.

### Performance

It may be desirable to provide a durable, light weight implantable prosthesis for breast reconstruction or augmentation without concern of long-term foreign material.

According to one aspect, the prosthesis may be fabricated from an absorbable material. In one embodiment, the prosthesis may be fabricated from a slow absorbing material, such as P4HB (Poly-4-hydroxybutyrate), to provide long term support for the breast as fat and/or tissue eventually fill and replace the prosthesis to promote a more natural appearance and feel for the breast. The material may be sufficiently porous to promote passage of fat and/or ingrowth of tissue within the prosthesis, although a porous material is not required for each embodiment. The prosthesis may include knitted, woven and/or non-woven material.

In one embodiment, the prosthesis may be fabricated with PHASIX mesh, which is manufactured from P4HB, available from Davol, Inc. of Warwick, RI. Other suitable materials may include, but are not limited to, GalaFLEX available from Galatea, TIGR Matrix available from Novus Scientific, SERI Surgical Scaffold available from Allergen, BIO-A available from Gore, and ULTRAPRO available from Ethicon. If desired, a non-woven material, such as Phasix, may be employed as an alternative or together with a mesh to provide a relatively softer profile for the prosthesis. For some applications, it may be desirable to fabricate the prosthesis or one or more portions of the prosthesis from a non-absorbable material including, but not limited to, polypropylene and polytetrafluoroethylene (PTFE).

For some applications, it may be desirable to coat the prosthesis with material to provide one or more properties. For example, and without limitation, it may be desirable to minimize bleeding, minimize seroma formation and/or facilitate tissue ingrowth. In one embodiment, the prosthesis may be coated with Arista AH available from Davol, Inc.

For some applications, it may be desirable for the prosthesis to have a relatively smooth outer surface. According to one aspect, the 3D structure of the prosthesis may be covered with an outer layer of material including, but not limited to, P4HB or collagen. For example, the prosthesis may include an outer layer that overlies the prosthesis body and that corresponds with the shape of the prosthesis body. As will be appreciated, an outer layer need not be required for each embodiment of the prosthesis. In other embodiments, one or more body segments may be arranged to have a relatively smooth outer surface and may have a smooth outer layer. In such embodiments, the outer layer may be applied to the body segment before attaching the body segment to the prosthesis body.

It may be desirable for particular applications for the prosthesis to be constructed to provide a desired amount of resistance to permanent deformation after compression. In one embodiment, the prosthesis may be constructed to have a reduction in height or no more than 10% (i.e., ≤10%) of its original height H after being subjected to vertical compression of 40% of its height H. The reduction in height may be determined at one or more times following compression. In one embodiment, the reduction in height may be determined at time t=0 and at time t=12 weeks. However, it is to be appreciated that the prosthesis may be configured to provide any suitable amount of resistance to permanent deformation as should be apparent to one of skill in the art.

It may be desirable to provide an implantable prosthesis which can support the resected space along with transferred adipose tissue during healing and integration of the implant.

According to one aspect, the prosthesis may have a compressive strength to oppose biomechanical forces within the breast. In one embodiment, the implant may have a compressive strength of at least 3.1 lbf (i.e., ≥3.1 lbf) at 25% vertical compression at time t=0 and at least 2.4 lbf (i.e., ≥2.4 lbf) at 25% vertical compression at time t=12 weeks. However, it is to be appreciated that the prosthesis may be configured to have any suitable amount of compressive strength as should appreciated by one of skill in the art.

According to one aspect, the prosthesis may employ connections having a connection strength which is sufficient to maintain the mechanical integrity of the device. In one embodiment, the implant may employ connections having a connection strength of at least 1.0 lbf (i.e., ≥1.0 lbf) at time t=0. However, it is to be appreciated that the prosthesis may be configured to have any suitable amount of connection strength as should appreciated by one of skill in the art.

The overall desired structure of the prosthesis may employ various constructs for its fabrication in an efficient manner.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

For purposes of this patent application and any patent issuing thereon, the indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. An implantable prosthesis comprising:
a tissue infiltratable body having a plurality of voids configured to receive fat and/or tissue, the body including a proximal end and a distal end spaced from the proximal end, the body including:
a plurality of three-dimensional (3D) body segments arranged in a stacked configuration along a longitudinal axis extending in a direction from the proximal end to the distal end of the body, wherein each of the 3D body segments include one or more outwardly facing voids.

2. The implantable prosthesis of claim 1, wherein each of the plurality of body segments is pleated or includes a honeycomb structure.

3. The implantable prosthesis of claim 1, wherein each of the plurality of 3D body segments is in direct contact with an adjacent 3D body segment.

4. The implantable prosthesis of claim 1, further comprising a plurality of two-dimensional (2D) body segments arranged in the stacked configuration with the plurality of 3D body segments.

5. The implantable prosthesis of claim 4, wherein each of the plurality of 2D body segments includes a planar sheet.

6. The implantable prosthesis of claim 4, wherein a first 3D body segment is stacked on top of a first 2D body segment.

7. The implantable prosthesis of claim 6, wherein the first 3D body segment is in direct contact with the first 2D body segment.

8. The implantable prosthesis of claim 7, wherein a second 3D body segment is stacked on top of and is in direct contact with the second 2D body segment.

9. The implantable prosthesis of claim 8, wherein the first and second 3D body segments are both pleated or both include a honeycombed structure.

10. The implantable prosthesis of claim 1, wherein a width of the body decreases in a direction from the proximal end to the distal end.

11. The implantable prosthesis of claim 1, wherein the longitudinal axis coincides with a central axis of the prothesis.

12. The implantable prosthesis of claim 1, further comprising a support structure coupled to the body and configured to support the body.

13. An implantable prosthesis comprising:
a tissue infiltratable body having a plurality of voids configured to receive fat and/or tissue, the body including a proximal end and a distal end spaced from the proximal end, the body including:
a plurality of three-dimensional (3D) body segments stacked on top of one another along a longitudinal axis extending in a direction from the proximal end to the distal end of the body, wherein each of the plurality of body segments is a toroidal shape;
wherein each of the plurality of body segments is in direct contact with an adjacent body segment.

14. The implantable prosthesis of claim 13, wherein each of the body segments includes an opening.

15. The implantable prosthesis of claim 14, wherein the openings of each of the plurality of body segments are aligned along the longitudinal axis to form a hollow core.

16. The implantable prosthesis of claim 13, wherein each of the 3D body segments is formed of a material having a plurality of pores defining a plurality of voids configured to receive fat and/or tissue.

17. The implantable prosthesis of claim 13, wherein the body includes a hub extending along a longitudinal axis, wherein the plurality of body segments is attached to the hub.

18. The implantable prosthesis of claim 13, wherein a width of the plurality of body segments decreases in a direction from the proximal end to the distal end.

19. The implantable prosthesis of claim 13, wherein the body is substantially semi-hemispherical in shaped.

20. The implantable prosthesis of claim 13, wherein the plurality of body components includes a first body segment and a second body segment, wherein the second body segment is placed within an opening of the first body.

21. The implantable prosthesis of claim 13, further comprising an outer layer defining a cavity, the body being located in the cavity.

22. The implantable prosthesis of claim 13, further comprising an outer layer that covers the body.

23. The implantable prosthesis of claim 13, further comprising a support structure coupled to the body and configured to support the body.

24. A method of fabricating an implantable prosthesis having a plurality of voids configured to receive fat and/or tissue, the method comprising acts of:
(a) stacking a first three dimensional (3D) body segment along a longitudinal axis of a body extending in a direction from a proximal end to a distal end of the body;
(b) stacking a first two-dimensional (2D) body segment along the longitudinal axis of the body;
(c) stacking a second 3D body segment along the longitudinal axis of the body; and
(d) attaching the first 3D body segment, the first 2D body segment, and the second 3D body segment together, wherein the first 2D body segment is positioned in between the first 3D body segment and the second 3D body segment.

25. The method of claim 24, wherein act (b) includes stacking a planar sheet along the longitudinal axis of the body.

26. The method of claim 24, wherein each of the first and second 3D body segments includes one or more voids for receiving fat and/or tissue.

27. The method of claim 24, wherein the first and second 3D body segments have the same configuration.

28. The method of claim 24, wherein the first and second 3D body segments have different configurations.

29. The method of claim 24, wherein act (d) includes suturing, gluing, and/or ultrasonically welding the first 3D body segment, the first 2D body segment, and the second 3D body segment together.

30. The method of claim 24, further comprising an act of (e) forming each of the first 3D body segment and the second 3D body segment.

31. The method of claim 30, wherein act (e) includes:
folding a first sheet of material in alternating directions along a plurality of fold lines to form a first folded sheet; and
folding first and second ends of the first folded sheet about a first axis to form a first circular fan arrangement.

32. The method of claim 31, wherein act (c) includes:
folding a second sheet of material in alternating directions along a plurality of fold lines to form a second folded sheet:
folding first and second ends of the second folded sheet about a second axis to form a second circular fan arrangement; and
attaching the second circular fan arrangement to the first circular fan arrangement.

33. The method of claim 30, wherein act (e) includes forming first and second 3D body segments having honeycomb structures.

34. The method of claim 30, wherein act (e) includes forming first and second 3D body segments having pleats.

35. The method of claim 30, wherein act (e) includes forming first a first and second 3D body segment having a toroidal shape.

36. The method of claim 24, wherein the body is configured to augment and/or reconstruct the anatomical shape of a human breast.
